(19) United States
(12) Plant Patent Application Publication
(10) Pub. No.: US 2004/0117879 P1
(43) Pub. Date: Jun. 17, 2004

(54) Miniature rose plant named 'BENfebu'

(75) Inventor: Frank Benardella, Englishtown, NJ (US)

Correspondence Address: Wendy R. White, Nor'East Miniature Roses, Inc., 58 Hammond St., P.O. Box 307, Rowley, MA, 01969-0607, US

(73) Assignee: Nor'East Miniature Roses, Inc., Rowley, MA

(21) Appl. No.: 10/318,843

(22) Filed: Dec. 13, 2002

(51) Int. Cl. A01H 5/00

(52) U.S. Cl. PLT/122

(57) ABSTRACT

‘BENfebu’ is a new and distinct variety of miniature rose plant primarily identified by its dark red flowers and petals of flushed white reverse, of hybrid tea form. There is little fade to the color of the flowers. Borne mostly in small clusters of 3 to 4, there are enough borne singly to use for exhibition cut flowers. The plant is upright, well branched and vigorous, clothed in dark green semi-glossy foliage.

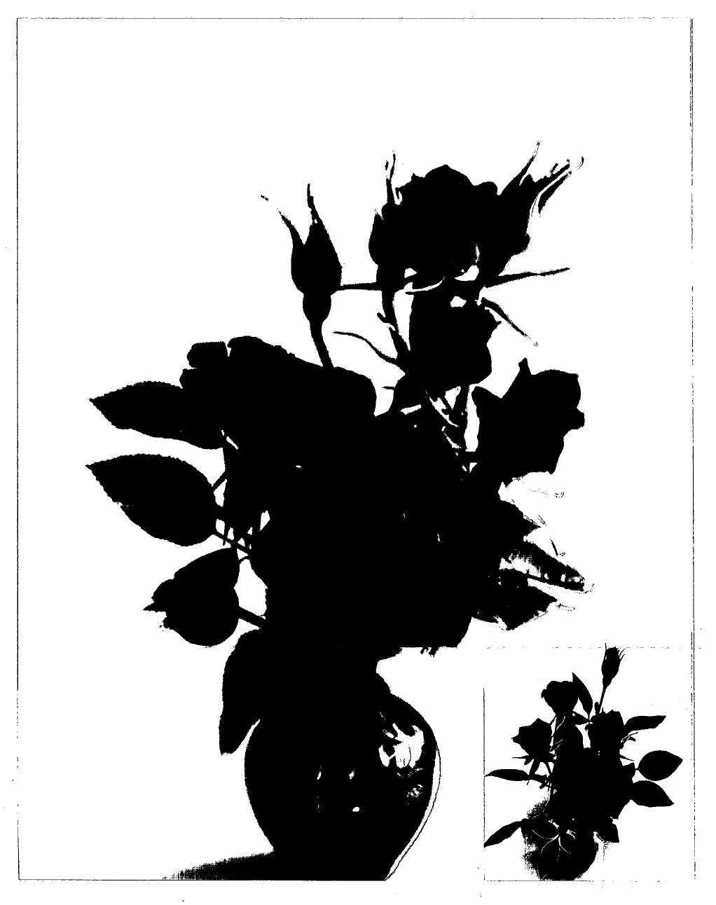

### CROSS REFERENCE

The present invention bares some resemblance to ‘BENmjul’ (U.S. Plant patent application Ser. No. 10/035,940 filed Dec. 21, 2001) from the same breeding program with which it shares some of the same parentage. More specifically, the seed parent of this new invention is ‘BENfig’ (U.S. Plant Pat. No. 8,020) which is a cross of ‘Rise ‘n’Shine’ (U.S. Plant Pat. No. 4,231) by ‘Laguna’ (not under patent protection) and the seed parent of ‘BENmjul’ is ‘BENjen’ (U.S. Plant Pat. No. 5,857) which is a cross of ‘Party Girl’ (U.S. Plant Pat. No. 4,598) by ‘Laguna’ and further that the seed parent of ‘Party Girl’ is ‘Rise ‘n’Shine’. Both cultivars have dark red buds and flowers of similar size and petal count on upright, well branched plants. There are 2 primary differences between BENmjul and the new cultivar. The new cultivar has more of its dark red flowers borne in clusters and that the reverse of these petals is flushed with white while on ‘BENmjul’ the dark red flowers are borne mostly singly on longer stems and the reverse of its petals is only a slightly lighter shade of red. Additionally, the anthocyanin coloration of the stems and foliage is mostly absent on this new invention while it is strong on BENmjul.

### RIGHTS TO THE INVENTION

Be it known that Frank A. Benardella of Englishtown, N.J., claims invention of new and useful improvements in ROSE PLANT/var. ‘BENfebu’ and following is a clear and exact description of the same.

### BACKGROUND OF THE INVENTION

Genus/species: Rosa hybrida ‘minima’

Varietal denomination: ‘BENfebu’

This present invention relates to a new and distinct variety of hardy, bush type plant of the miniature rose class and with exhibition form, the primary objective of this breeding program. This new variety was created in a green-house in Englishtown, N.J. by crossing the following two rose plants:

The seed parent is ‘BENfig’ (U.S. Plant Pat. No. 8,020).

The pollen parent is an unnamed, unintroduced miniature rose (not patented) which was a cross of ‘KINfancy’ (not patented) by ‘BENblack’ (U.S. Plant Pat. No. 5,925).

This present invention bares resemblance to its seed parent, BENfig (U.S. Plant Pat. No. 8,020), with a similar bud shape and petal count and size. The differences between those two plants are that the flowers of this new invention are a dark red with a lighter reverse as compared to light pink flowers with a lighter reverse on the seed parent and this new invention has a more compact plant habit. The primary trait from it's pollen parent is its red coloring.

Asexual reproduction by cuttings of this new variety in Englishtown, N.J. and Rowley, Mass. shows that all distinguishing characteristics of this rose continually come true to form.

### SUMMARY OF THE INVENTION

The present invention relates to a new and distinct variety of hardy dwarf bush type rose plant of the miniature class; the variety being primarily characterized—as to novelty—by it's small, hybrid tea form, dark red blooms flushed white on the reverse.

The variety is further characterized by:

An abundance of these flowers borne mostly in small clusters of 3 or 4 but also singly

Semi-glossy foliage

A slight rose fragrance

A plant which has good vigor, is upright and well branched.

A plant which grows and blooms satisfactorily both in the greenhouse and outdoors; provides decoration as a garden perennial, specimen plant or in large containers and is good for cut flowers and exhibition.

### BRIEF DESCRIPTION OF THE DRAWING

The accompanying drawing is of 2 color photographs showing specimens of the buds, flowers and foliage of this new variety in different stages of development as grown under plastic in Essex County, Massachusetts. The larger photograph was taken in October and shows one hip nearing maturity. (Darkened areas on the edges of some of the petals was caused by stress to the plant by inferior watering practices, prior to the taking of this picture.) The inset photograph was taken in August and shows the greater intensity of the white flushing the reverse of the petals as noted on this plant throughout the summer months.

### BOTANICAL DESCRIPTION OF THE PLANT

Following is a detailed description in outline form pertaining specifically to this new and distinct variety of miniature rose plant as observed during the summer months at between 4 and 5 years of age, growing indoors, under plastic in an 8 inch standard nursery container, in Essex County, Massachusetts. All major color plate identifications made are referring to The Royal Horticulture Society Chart except where common terms of color definition are employed.

Genus/species: Rosa hybrida ‘minima’.

Varietal denomination: ‘BENfebu’.

Commercial class: Miniature Rose.

Seed parent: ‘BENfig’ (U.S. Plant Pat. No. 8,020).

Pollen parent: a seedling which was a cross of ‘KINfancy’ (not patented) by ‘BENblack’ (U.S. Plant Pat. No. 5,925).

### FLOWERS

Blooming habit: recurrent.

Borne: Mostly in small clusters of 3 or 4 flowers, but also singly.

Bud:

Size.—11/16 to 15/16 of an inch in length and ½ inch in diameter at the widest point just before the sepals divide.

Form.—narrow ovate.

Sepals.—color — an olive green, near 146B and inside surface lighter, near 147C and with 147B along the margins of the interior. size — 1 to 1 1/16 inch long and 5/16 inch wide excluding any foliation; extending ½ to ¾ inch beyond the tip of the bud just before they start to divide characteristics — lanceolate; 2 innermost sepals with smooth edges and the 3 outermost sepals are very finely beaded with 1 to 3 narrow, foliar appendages on one or both edges; rolling back first to a 90 degree angle with the receptacle; as the petals start to unfurl, rolling back almost to the stem and as the hip forms and matures returning to and remaining at a 90 degree angle with the receptacle surface texture — inside surface pubescence; the outer surface is glaborous.

Peduncle.—length — averaging ⅞ inch aspect — straight strength — strong, erect color — yellow green, near 144B, anthocyanin coloration absent texture — usually with a medium amount of stiff hairs.

Receptacle.—diameter — about 5/16 inch color — near 144A texture — glaborous.

Color.—When sepals first divide — a medium red, near 57B.

Bloom:

Size.—When fully expanded — 2 inches.

Form.—High centered at first with petals reflexing slightly as they start to unfurl and when full open having a flattened convex upper profile and a flat lower profile with petals remaining only slightly reflexed.

Petalage.—23 to 28.

Petaloids.—1 or up to 7.

Fragrance.—slight.

Persistence.—usually drops off cleanly in 6 to 10 days.

Lasting quality as cut flower.—4 to 8 days.

Petals:

Texture.—glaborous.

Appearance.—Inside — velvety, outside — satiny.

Form.—broad fan shaped, rounded base; margins with a very slight undulation and usually without a definitive apex on the outer petals and sometimes a very minute serration near the center of the outer margin on some of the inner petals; reflexing of margins is slight.

Length and width in inches.—as wide as they are long — 1 inch by 1 inch to 1⅛ inches by 1⅛ inches.

Arrangement.—Arranged shingle-like.

Color.—during the first few days — inner and outer petals are a dark cardinal red, with inner petals being between 53A and 46A and outer petals being between 53B and 46B; the reverse is flushed with white giving the appearance of being near 66C as in the smaller inset image in the accompanying drawing and occasionally with one or two of the petals having a streak up through their center, which is a very pale yellow, near 4D When half blown — both inner and outer petals are between 53B and 46B and the reverse remaining near 66C Seasonal differences — in October, the reverse of the petals is only lightly flushed and always being near 53C, as depicted in the larger of the accompanying images; the upper surface color is very near the same as in the summer with no visible difference Variegations — although the upper surface of most of the petals is a solid cardinal red some of the inner petals may have a streak of very light yellow, near 4D, up through the center. When there are streaked petals within a flower, there is usually 3 or more but never more than 7 petals with the streak and they are not usually adjacent to one another. As previously noted, some of the outer petals may have the very light streak going up the middle of the reverse. Base of petals — near 4D and going about ⅕ of the way up the petals. Petals at point of attachment — near 6D As the bloom ages there is little change in the color as it lightens only slightly, remaining near 53B.

General tonality.—dark red.

Petaloids:

Color.—same as that of the inner petals except there is apt to be more of the very light yellow, near 4D, streaking up from the base to the tip.

Size.—width — ¾ inch at the widest point length — quite variable from ¼ inch to 7/16 inch.

### REPRODUCTIVE ORGANS

Stamens, filaments and anthers:

Arrangement.—regularly arranged around styles.

Quantity.—120 to 130.

Filaments.—length — varying 4/32 to 11/32 inch general color — light yellow, near 8C.

Anthers.—color — light yellow but slightly darker than the filaments, near 10C.

Pollen:

Color.—a medium orange yellow, near 21C.

Quantity.—ample.

Pistils, styles and stigmas:

Quantity.—about half or a few less than half as many as stamens.

Styles.—thin; uneven length between ⅛ and 2/8 inch color — translucent, light yellow green at their base, near 2D, and becoming just a light translucent yellow, near 11D upward from the base.

Stigmas.—color — a light yellow, just a shade darker than the upper portion of the styles, near 8D.

Hips:

Shape.—pumpkin shaped, smooth surface; seeds are entirely enclosed within the hip.

Size.—usually 11/16 inch in diameter.

Color.—becoming near 169B from the grayed-orange group.

Seed.—color — a light “grayed” yellow, between 162C and 162D size — averaging 5/32 inch diameter, obovate shape and irregular sides.

### PLANT

Habit: upright and well branched.

Growth: vigorous and uniformly branched, maturing at a height between 26 and 34 inches and a width of 30 to 36 inches.

Length of flowering stem: averaging 6 to 9 inches when borne singly.

Foliage: near pinnately compound (some leaflets, most often the basil ones, are not directly opposite by as much as 1/32 inch) with 5 leaflets, occasionally with 3 or 7.

Size of mature leaf.—3½ to 3¼ inches from plant stem to tip when measured along the rachis; mature terminal leaflet — 1¼ to 1⅞ inches from base to tip and 13/16 to 15/16 inch wide at it's widest part.

Quantity.—abundant.

Leaflets.—shape — ovate, tapering towards both ends, to an acuminate apex and a narrow oval base color — new foliage, upper surface, 137A, under surface near 137B; older foliage — a very dark green, near 147A, on the upper surface and a medium green, near 147B, on the reverse; anthocyanin coloration is absent appearance — upper surface, semi-glossy; underside, matte with veins protruding slightly of a light olive green, near 144C, texture — both surfaces are glaborous, under surface — lightly textured from veins and leathery edge — fine, near even serration and with stipate glands unevenly spaced along the entire margin, often on the outer point of the serration.

Petiole/Rachis.—infrequently having 1 to 3 undeveloped basil leaflets which sometimes appear as tiny leaflets and other times as very tiny appendages, not always evenly spaced or opposite color on old leaf — upper side is an dark olive green, between 147A and 146A, underside near 144A color on young leaf — upper side is a medium green, near 137C; the underside is near 144A texture — moderately smooth; upper side is shallowy grooved with some stipate glands on the edges of the grooves; the underside is very smooth with a few stipate glands and occasionally 1 to 3 prickles. rachis — length varies from 2⅝ to 2 5/16 inch.

Petiolules.—color — on old leaf, upper side is a lighter olive green than the rachis, near 147C, and the underside is also lighter, near 144C; on young leaf, the color is the same as that of the petiole/rachis on both surfaces. size — usually 1/32 of an inch long but sometimes even shorter to the point of appearing non existent.

Stipules.—very narrow, measuring 10/16 to 11/16 inch in length; margin — fine and uneven serirulation and the very edge tightly reflexing on the mature leaf.

Resistance: good resistance to downy and powdery mildews and insects; average resistance to blackspot; no rust observed.

Wood:

New wood.—texture — smooth, glaborous color — near 146A.

Old wood.—color — between 147B and 146C and having areas of and then becoming entirely covered with very slightly raised and corky textured surface and near 199B.

Thorns:

Quantity.—on main stalks, few and on laterals, ordinary.

Form.—an elliptical base tapering to a fine point, straight and angled slightly down, being ¼ to 3/16 inch in length.

Color when young.—a medium shade of yellow green, near 151A.

Color when old.—near 164B from the greyed-orange group.

Prickles.—1 to 3 occasionally found on underside of the rachis only color: near 19D, Egyptian buff yellow.

Winter hardiness: Tested hardy in zones 4 through 9 with ample winter protection recommended zones 6 and colder.

1. A new and distinct variety of hardy miniature rose plant is claimed, substantially as illustrated and described, characterized by dark red, non-fading blooms of hybrid tea form, showing a lighter reverse before it is full open on an upright, vigorous plant.
